(19) United States
(12) Patent Application Publication
(10) Pub. No.: US 2022/0265992 A1
(43) Pub. Date: Aug. 25, 2022

(54) LEFT VENTRICULAR ASSIST SYSTEM AND METHOD OF DRIVING BLOOD PUMP

(71) Applicant: SUN MEDICAL TECHNOLOGY RESEARCH CORPORATION, Nagano (JP)

(72) Inventors: Nozomi KOIKE, Nagano (JP); Masahiko ITO, Nagano (JP); Shinji KOBAYASHI, Nagano (JP)

(21) Appl. No.: 17/679,097

(22) Filed: Feb. 24, 2022

(30) Foreign Application Priority Data
Feb. 25, 2021 (JP) 2021-028348

(51) Int. Cl. A61M 60/216; A61M 60/126

(52) U.S. Cl. CPC A61M 60/216; A61M 60/126

(57) ABSTRACT

Provided is a left ventricular assist system that includes at least: a blood pump; and a controller that controls the rotation of a rotary body of the blood pump. The controller controls the blood pump such that a rotational speed of the rotary body is periodically switched between a first rotational speed and a second rotational speed that is larger than the first rotational speed with a transition time required for switching the rotational speed set to substantially 0 (zero). Periodic switching of the rotational speed of the rotary body is asynchronous with a cardiac cycle of a user, and the rotary body rotates substantially only at rotational speeds of two values consisting of the first rotational speed and the second rotational speed.

### BACKGROUND OF THE INVENTION

### Field of the Invention

The present invention relates to an implantable left ventricular assist system and a method of driving a blood pump.

### Description of the Related Art

A left ventricular assist system is a system that assists delivery ability of blood delivered from a left atrium to an aorta. The left ventricular assist system is used to assist the circulation of blood of a patient (user) whose heart has a weak pumping function. Recently, extensive studies have been made in this field, and a so-called “implantable left ventricular assist system”, a type of ventricular assist system where a blood pump is embedded in a human body is now available.

To introduce one example of a left ventricular assist system, the left ventricular assist system includes: a continuous-flow-type blood pump that sucks and delivers blood by rotating an impeller (rotary body); a blood removal tube having an upstream side that is connected to a left ventricle and a downstream side that is connected to a suction port of the blood pump; a blood delivery tube having an upstream side that is connected to a delivery port of the blood pump and a downstream side that is connected to an aorta; a controller that controls an operation of the blood pump and the like. In a normal operation mode, to assist the circulation of blood, the left ventricular assist system is operated in general by rotating an impeller of the blood pump at a fixed rotational speed (see the website of the general incorporated association The Japanese Society For Artificial Organs “ventricular assist device”, [online], [retrieved on Jan. 21, 2021], Internet (URL: https://www.jsao.org/public/what/what03/) and see the website of the general incorporated association The Japanese Circulation Society “Guidelines for Device Therapy Implantable Left Ventricular Assist Device for Patients with Severe Heart Failure (JCSJSCVS2013)”, [online], [retrieved on Jan. 21, 2021], Internet (URL: https://www.jcirc.or.jp/old/guideline/pdf/JCS2013_kyo_h.pd f))

### SUMMARY OF THE INVENTION

However, in a case where pulsation (heartbeat) of a user's own heart are originally weak, even when the user receives a blood circulation assist by a blood pump, a pressure difference between a left ventricle and an aorta (difference between the pressure in the aorta and the pressure in the left ventricle) induced by the pulsation of user's own heart is small. Accordingly, a large open-close operation (opening and closing over a full movable range) of an aortic valve cannot be expected. Further, a change in an inner pressure in the left ventricle and a change in an inner pressure in an aorta vessel are also small, the stretching and contraction of tissues in the vicinity of the aortic valve such as an inner wall of the aorta and a proximal portion of the aorta cannot also be expected. Accordingly, blood in the vicinity of the aortic valve is liable to be locally stagnated.

There may be a case where, among users who have weak pulses, some users have a failure in an aortic valve per se (an aortic valve stenosis, an aortic regurgitation or the like). In this case, opening and closing of the aortic valve are not performed over a full movable range and hence, blood in the vicinity of the aortic valve is swirled so that blood is liable to be locally stagnated.

Further, there may also be a case where, among users who have weak pulsation, some users have a failure in a mitral valve per se (mitral valve regurgitation or the like). For example, in a case of the mitral valve regurgitation, a mitral valve is opened half in an ejection period and hence, a pressure loss occurs in the left ventricle whereby there is a concern that the aortic valve is not sufficiently opened. As a result, there arises a possibility that blood in the vicinity of the aortic valve locally stagnates.

In any case, the stagnation of blood is not desirable because the stagnation of blood causes a thrombus. Accordingly, there has been a demand for a left ventricular assist system where the stagnation of blood minimally occurs.

The presence of “pulses” in circulation of blood in a human body is important for a viewpoint of realizing a function of delivering arterial blood to a systemic circulation system such as various organs, a brain, terminals of four limbs and the like. In addition to such a function, due to the presence of “pulses”, even at the destinations to which blood is delivered, a physiological phenomenon that is favorable for a human to maintain a health (a physiological phenomenon expected by pulses) can be realized. For example, a pressure in a blood vessel is changed in a pulsating manner in association with the pulsation and hence, tissues that form a blood vessel are stretched and contracted whereby an inner diameter of the blood vessel is changed.

In a case where the heartbeat of a patient (user) is originally weak or pulses of the patient are weak because of other causes, it is considered that the patient cannot sufficiently enjoy the above-mentioned physiological phenomenon expected by pulses.

The present invention has been made in view of the above-mentioned circumstance, and it is an object of the present invention to provide a left ventricular assist system having the simple configuration that can minimize the stagnation of blood, and enables a user to enjoy a “physiological phenomenon expected by pulses” even in a case where pulses of an own heart of the user are originally weak. It is another object of the present invention to provide a method of driving a blood pump of such a left ventricular assist system.

According to an aspect of the present invention, there is provided an implantable left ventricular assist system.

The left ventricular assist system includes as least: a continuous-flow-type blood pump configured to suck and deliver blood by rotation of a rotary body housed in a pump chamber; and a controller connected to the blood pump and configured to control the rotation of the rotary body of the blood pump.

In a normal operation mode, the controller is configured to control the blood pump such that a rotational speed of the rotary body is periodically switched between a “first rotational speed” and a “second rotational speed” that is larger than the first rotational speed with a transition time required for switching the rotational speed set to substantially 0 (zero). Further, in the left ventricular assist system, periodic switching of the rotational speed of the rotary body is asynchronous with a cardiac cycle of a user of the left ventricular assist system, and the blood pump is operated so as to make the rotary body rotate substantially only at rotational speeds of two values consisting of the first rotational speed and the second rotational speed.

According to the left ventricular assist system of the present invention, the system can minimize the stagnation of blood while having the simple configuration. Further, the system enables a user to enjoy a “physiological phenomenon expected by pulses” even in a case where the pulses of an own heart of the user are originally weak.

According to another aspect of the present invention, there is provided a method of driving a blood pump in a left ventricular assist system that includes a continuous-flow-type blood pump configured to suck and deliver blood by rotation of a rotary body housed in a pump chamber.

In the method of driving a blood pump, in a normal operation mode, the blood pump is operated such that a rotational speed of the rotary body is periodically switched between a “first rotational speed” and a “second rotational speed” that is larger than the first rotational speed with a transition time required for switching the rotational speed set to substantially 0 (zero). In the method, in the left ventricular assist system, periodic switching of the rotational speed of the rotary body is asynchronous with a cardiac cycle of a user of the left ventricular assist system, and the blood pump is operated so as to make the rotary body rotate substantially only at rotational speeds of two values consisting of the first rotational speed and the second rotational speed.

According to the method of driving a blood pump of the present invention, the left ventricular assist system can minimize the stagnation of blood while having the simple configuration. Further, the method enables a user to enjoy a “physiological phenomenon expected by pulses” even in a case where an own heart of the user is originally weak.

### BRIEF DESCRIPTION OF THE DRAWINGS

FIG. 1 is an overall view of a left ventricular assist system according to an embodiment;

FIG. 2 is a cross-sectional view for describing a blood pump 10 according to the embodiment;

FIG. 3 is a schematic cross-sectional view described focusing on a flow path of blood in the left ventricular assist system attached to the own heart;

FIG. 4 is a block diagram for describing a controller according to the embodiment;

FIG. 5 is a timing chart describing switching of a rotational speed of a rotary body of the blood pump; and

FIG. 6 is a flowchart for describing a method of driving a blood pump according to the embodiment.

### DETAILED DESCRIPTION OF THE PREFERRED EMBODIMENTS

Hereinafter, a left ventricular assist system 1 and a method of driving a blood pump according to the present invention are described with reference to attached drawings. The respective drawings are schematic drawings that describe one example of the left ventricular assist system 1 and the method of driving a blood pump, and do not always strictly reflect actual sizes of constitutional elements, actual ratios between the sizes of constitutional elements, and the like.

### Embodiment

### 1. Configuration of Left Ventricular Assist System 1 According to Embodiment

### (1) Overall Configuration

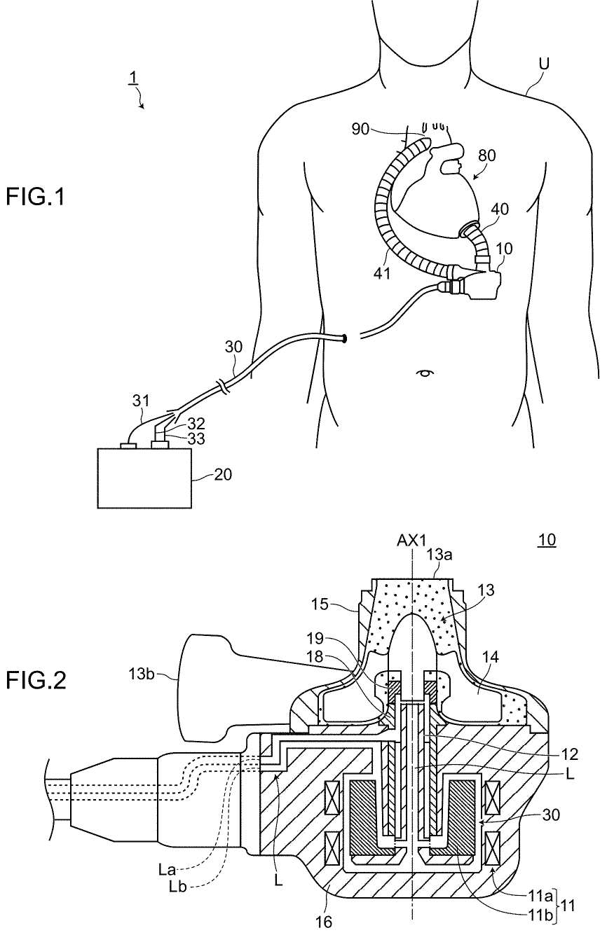

FIG. 1 is an overall view of a left ventricular assist system 1 according to an embodiment.

As illustrated in FIG. 1, the left ventricular assist system 1 according to the embodiment (this system) is an implantable left ventricular assist system that includes: a blood pump 10; artificial blood vessels 40, 41 that connect the blood pump 10 and organs (own heart 80, aorta 90) of a user to each other; a controller 20 that controls an operation of the blood pump 10; and a pump cable 30 that electrically connects the blood pump 10 and the controller 20 to each other. In this embodiment, “implantable” means that the left ventricular assist system 1 is of a type where the blood pump 10 is embedded in a user U.

### (2) Blood Pump 10

FIG. 2 is a cross-sectional view for describing the blood pump 10 according to the embodiment.

As illustrated in FIG. 2, the blood pump 10 is a continuous-flow-type blood pump where blood is sucked from a suction port 13a and is delivered from an ejection port 13b when an impeller 14 that forms a rotary body housed in a pump chamber 13 that is surrounded by a casing 15 and a base body 16 is rotated about a rotation axis AX1.

Assuming the blood pump 10 as a path through which blood passes, no particular constitutional element such as a valve is not disposed between the suction port 13a and the ejection port 13b of the pump chamber 13 so that the inside of the pump chamber 13 forms a continuous space. In this specification, it should be construed that the “continuous-flow-type” pump is a concept that embraces a centrifugal pump, an axial pump and the like but excludes a diaphragm-type pump and the like.

In this embodiment, as the rotary body, the impeller 14 having the structure illustrated in FIG. 2 is exemplified. However, the rotary body may have other structures. In the description made hereinafter, the rotary body may be referred to as the impeller 14, and the impeller 14 may be referred to as the rotary body. These terms are convertible.

As a device that drives the impeller 14, a motor 11 (having a stator 11a and a rotor 11b) is disposed on a base body 16 side. A shaft 12 is connected to the rotor 11b of the motor 11, and the impeller 14 is connected to the shaft 12 by way of a seal ring 19. Rotary axes of the rotor 11b, the shaft 12, the seal ring 19 and the impeller 14 are coaxial with the rotation axis AX1. When the motor 11 (rotor 11b) is rotated, the impeller 14 is integrally rotated with the motor 11 about the rotation axis AX1.

To describe other constitutional elements for a reference purpose, the seal ring 19 is brought into contact with a seat ring 18 fixed to a non-rotational part of the base body 16. When the seal ring 19 is rotated, sliding takes place between the seal ring 19 and the seat ring 18. A cooling and sealing liquid is supplied to areas in the vicinity of the shaft 12, the motor 11 and the like besides such a sliding portion (not illustrated in the drawing) through a cooling and sealing liquid flow path L. The cooling and sealing liquid circulates in the blood pump 10 such that the cooling and sealing liquid is supplied from a storage tank of the controller 20 to the sliding portion, the shaft 12 and the like through a rising tube 32 (see FIG. 1) and a rising flow path La (see FIG. 2), and the cooling and sealing liquid is returned to the controller 20 from the sliding portion, the shaft 12 and the like through a lowering flow path Lab and a lowering tube 33. Accordingly, the pump cable 30 includes the rising tube 32 and the lowering tube 33 through which a cooling and sealing liquid flows besides an electric cable 31 that performs electrical connection.

In this embodiment, it is not always necessary for the blood pump 10 to include such a system that circulates a cooling and sealing liquid.

### (3) Auxiliary Route 55 and Original Route 95 of Blood Flow

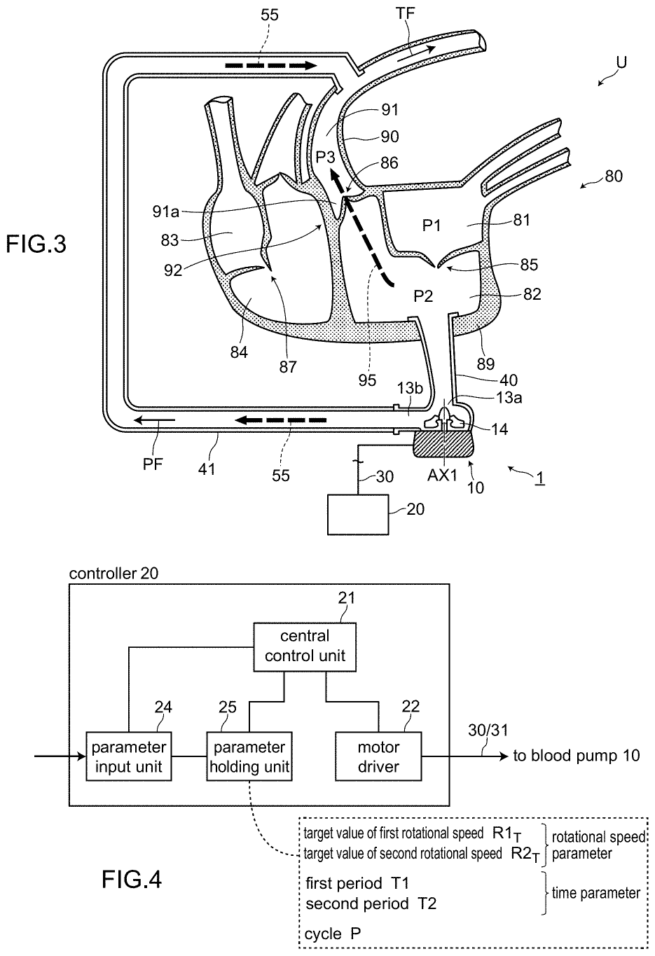

FIG. 3 is a schematic cross-sectional view described focusing on a flow path of blood with respect to the left ventricular assist system 1 attached to the own heart 80.

As illustrated in FIG. 3, when the left ventricular assist system 1 is attached to the own heart 80, an upstream side of the artificial blood vessel 40 (blood removal tube) is connected to a left ventricle 82 of the own heart 80 endermically, a downstream side of the artificial blood vessel 40 (blood removal tube) is connected to the suction port 13a of the blood pump 10, an upstream side of the artificial blood vessel 41 (blood delivery tube) is connected to the ejection port 13b of the blood pump 10, and a downstream side of the artificial blood vessel 41 (blood delivery tube) is connected to the aorta 90 endermically.

A portion of blood of a user flows through an “auxiliary route 55” that traces the artificial blood vessel (blood removal tube), the blood pump 10 and the artificial blood vessel 41 (blood delivery tube). As a term that is compared with the auxiliary route 55, a route where blood flows from the left ventricle 82 of the own heart 80, passes an area in the vicinity of an aortic valve 86, and reaches the aorta 90 is referred to as a “original route 95”.

A flow rate of blood that flows the auxiliary route 55 is referred to as a pump flow rate PF, and a flow rate that is a sum of a flow rate of blood that flows the original route 95 and the pump flow rate PF of blood that flows the auxiliary route 55 is referred to as a total flow rate TF.

Symbol 81 indicates a left atrium, symbol 83 indicates a right atrium, symbol 84 indicates a right ventricle, symbol 87 indicates a tricuspid valve, symbol 88 indicates a pulmonary valve, symbol 89 indicates a cardiac muscle, symbol 91a indicates an area in the vicinity of an aortic valve in the inside of the aorta, and symbol 92 indicates an aortic root. With respect to symbols that are also used in FIG. 1 and FIG. 2 in common, the contents that are already described with reference to the symbols are used and are not described here.

### (4) Controller 20

FIG. 4 is a block diagram for describing the controller 20 according to the embodiment.

The controller 20 is electrically connected to the blood pump 10 via the pump cable 30 (specifically, the electrical cable 31), and controls the rotation of the impeller 14 that is the rotary body of the blood pump 10. The concept “controls the rotation” includes: a control of the rotary body (impeller 14) so as to make a rotational speed of the rotary body reach a desired target amount; a control of a period during which a desired rotational speed is maintained; changing a rotational speed from a present rotational speed to a desired rotational speed; and a control of changing switching timing of a rotational speed, and the like.

As illustrated in FIG. 4, the controller 20 includes: a central control unit 21; and a parameter inputting unit 24, a parameter holding unit 25, and a motor driver 22 that are connected to the central control unit 21.

The central control unit 21 is a part that mainly controls an entire operation of the blood pump 10. For example, a suitable processor can be used as the central control unit 21.

The motor driver 22 is electrically connected to the motor 11 of the blood pump 10. The motor driver 22 controls the motor 11 in response to a command from a host control unit (the central control unit 21 in this embodiment).

As a command from the host control unit, for example, a target amount relating to a speed of the motor (a rotational speed of the motor as an example) can be named. For example, when the central control unit 21 writes a rotational speed of the motor 11 that is a target in a register (not illustrated in the drawing) having a predetermined address in the form of digital data, the motor driver 22 is instantaneously operated so as to change a rotational speed of the motor 11 and to rotate the motor 11 at a rotational speed that is a target amount by referencing the content of the register.

In this embodiment, writing a rotational speed of the motor 11 that is a target in the register having a predetermined address by the central control unit 21 in the form of digital data has the same meaning as “commanding” a rotational speed, issuing a “command” of a rotational speed, outputting a “control signal” of a rotational speed and the like.

The parameter inputting unit 24 and the parameter holding unit 25 are described later.

### (5) Rotation Control of Motor 11 (Rotary Body) by Controller 20

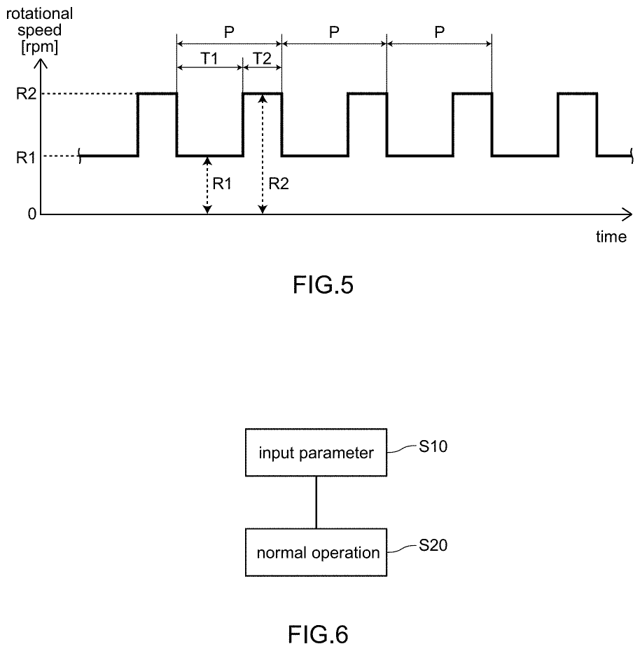

FIG. 5 is a timing chart describing switching of a rotational speed of the rotary body (impeller 14) of the blood pump 10. Time is taken on an axis of abscissas, and a rotational speed of the impeller 14 is taken on an axis of ordinates. In this embodiment, “rotational speed” means the number of times that the rotary body rotates per a unit time. In the drawing, a unit “rpm (revolution per minute)” is used for the sake of convenience.

(5-1)

In a normal operation mode, the left ventricular assist system 1 according to this embodiment is configured to rotate the impeller 14 at complete rotational speeds of two values as illustrated in FIG. 5, for example. In this embodiment, the “normal operation mode” means a normal operation mode excluding non-normal operation modes such as an adjustment operation of the blood pump 10, a rising operation in accordance with a sequence immediately after supplying electricity to the blood pump 10 or a battery exchange operation at the time of exchanging a battery in the controller 20.

As illustrated in FIG. 5, in a normal operation mode, the controller 20 is configured to control the blood pump 10 such that a rotational speed of the rotary body (impeller 14) is switched periodically between a “first rotational speed R1” and a “second rotational speed R2” that is larger than the first rotational speed R1 with a transition time required for switching the rotational speed set to substantially 0 (zero). In other words, the controller 20 is configured to operate the blood pump 10 such that the impeller 14 is rotated substantially only at the rotational speeds of two values consisting of the first rotational speed R1 and the second rotational speed R2.

In FIG. 5, a timing chart of a cardiac cycle of the user U is not illustrated. However, periodic switching of a rotational speed of the rotary body illustrated in FIG. 5 is asynchronously performed with a cardiac cycle of the user U of the left ventricular assist system 1.

In this embodiment, a period during which the impeller 14 is rotated at the first rotational speed R1 is defined as a “first period T1”, a period during which the impeller 14 is rotated at the second rotational speed R2 is defined as a “second period T2”, and a sum of the first period T1 and the second period T2 is defined as one cycle P.

(5-2)

A switching time between the first period T1 and the second period T2, that is, the transition time necessary for switching a rotational speed is substantially 0 (zero).

It also implies the following. In the control of the motor 11 of the blood pump 10, in instructing a rotational speed to the motor driver 22 (in issuing a command of a rotational speed to the motor driver 22), when the rotational speed is switched from the first rotational speed R1 to the second rotational speed R2 or the rotational speed is switched from the second rotational speed R2 to the first rotational speed R1, an intermediate rotational speed between the first rotational speed R1 and the second rotational speed R2 is not instructed (a command is not issued). In other words, to consider the control of the blood pump 10 on the basis of the instruction of a rotational speed (issuing of a command), a transition time is 0 (zero), an instruction (command) of a rotational speed is issued discretely at two values consisting of the first rotational speed R1 and the second rotational speed R2, and an instruction (issuing of a command) of a rotational speed that allows the motor 11 of the blood pump 10 to perform ramp driving that requires a transition time in the middle of the driving and has a speed gradient is not issued.

In actually building the system and operating the system, even when an instruction (command) that discretely changes a rotational speed is issued to the motor driver 22 from the central control unit 21, because of the presence of an electric response delay time in the motor driver 22, the presence of a response delay time due to a mechanical factor of the motor 11, the presence of a response delay time attributed to a load brought about by blood that is in contact with the impeller 14 or the like, a time required for changing a rotational speed of the impeller 14 (a transition time required for switching a rotational speed) cannot be set to 0 (zero) in a strict meaning of the term 0 (zero).

However, even when a transition time required for switching a rotational speed of the impeller 14 is eventually slightly generated due to such a response delay, so long as an instruction (issuing of a command) is discretely issued at two values consisting of the first rotational speed R1 and the second rotational speed R2, and an intermediate speed is not instructed (a command is not issued) in the midst between the first rotational speed R1 and the second rotational speed R2, such a state is determined to satisfy the definition “a transition time required for switching a rotational speed is substantially 0 (zero)” and “the rotary body rotates substantially only at rotational speeds of two values consisting of the first rotational speed R1 and the second rotational speed R2”.

For example, when this system is carried out using a technique at the time of filing the present application, to consider the control of the blood pump 10 on the basis of a rotational speed of the impeller 14, there may be a case where a transition time becomes 40 ms or less, for example. So long as the transition time is 0 (zero) on the basis of an instruction (issuing of a command) of a rotational speed, such a case is also defined that a transition time required for switching a rotational speed of the impeller 14 is substantially 0 (zero).

(5-3)

In the above-mentioned embodiments, parameters such as the first rotational speed R1, the second rotational speed R2, the first period T1, the second period T2, the one cycle P, a duty ratio of the second period T2 in the one cycle P can be set as desired. However, it is considered that, depending on a condition of a user U, by setting these parameters to values close to behaviors of a healthy heart of a person in general, a predetermined favorable effect can be acquired.

For example, the user U of the left ventricular assist system 1 is at rest, a value of the one cycle P can be set such that the number of appearances of the second period T2 becomes a predetermined number within a range of 40 times to 100 times per minute that is substantially as same as the number of pulses of a healthy person. Preferably, the one cycle P can be set such that the number of appearances of the second period T2 becomes the predetermined number that falls within a range of 50 times to 80 times per minute.

Further, for example, to realize a duty ratio of the second period T2 in the one cycle P (that is, T2/P) substantially equal to a duty ratio in an ejection period with respect to a cardiac cycle of a healthy person, that is, to set a duty ratio of the second period T2 in the one cycle P (that is, T2/P) such that the duty ratio falls within a range of 0% to 40%, the values of the first period T1, the second period T2 and the one cycle P can be set. Preferably, these parameters are set such that the duty ratio falls within a range of 30% to 35%.

Further, for example, it is said that a flow rate of blood in the aorta 90 of a healthy person takes a value that falls within a range of 10 L to 30 L per minute as a peak flow amount.

In view of the above, the second rotational speed R2 can be set to a rotational speed obtained by calculating in a reverse manner by subtracting a function of the own heart 80 such that a blood flow (the total flow rate TF in FIG. 3) in the aorta 90 of the user U of the left ventricular assist system 1 falls within a range of 10 L to 30 L per minute as a peak flow amount. Preferably, the second rotational speed R2 is set such that the blood flow has a peak flow amount of the total flow rate TF that falls within a range of 25 L to 30 L per minute.

In setting a desired peak flow amount of a blood flow in the aorta 90, the peak flow amount can be set corresponding to “user attribute information” such as a weight, a height, an age, a peak flow amount measured during a healthy time or the like, for example.

For example, it is said that the total flow rate TF of blood in an aorta in an isovolumic relaxation or in an isovolumic contraction of a healthy person becomes substantially in the vicinity of 0 (zero).

Based on this understanding, the second rotational speed R2 can be set to a rotational speed by taking into account a desired peak flow amount of a blood flow (the total flow rate TF) in the aorta 90 of the user U of the left ventricular assist system 1, and the first rotational speed R1 can be set to a rotational speed obtained by calculating in a reverse manner by subtracting a function of the own heart 80 such that a blood flow (the total flow rate TF) in the aorta 90 of the user U takes a value in the vicinity of 0 (zero).

### (6) Variability of Rotational Speed Parameters and Time Parameters

The above-mentioned parameter can be suitably varied even after this system is embedded into the user U in an assembled manner.

As illustrated in FIG. 4, the controller 20 includes the parameter input unit 24 and the parameter holding unit 25.

Into the parameter input unit 24, at least one parameter out of a target amount R1_{T} of the first rotational speed and a target amount R2_{T} of the second rotational speed that form “rotational speed parameters” given from the outside and at least one parameter out of the first period T1, the second period T2 and one cycle P (further possibly including a duty ratio of the second period T2 with respect to the one cycle P and the like) that form “time parameters” are inputted respectively. Some parameters may be preliminarily held in the parameter holding unit 25 as fixed values, and the remaining parameters may be formed such that these parameters are given as variable parameters from the outside.

The parameter input unit 24 may be formed of, for example: a key switch (not illustrated in the drawing) that is configured to allow key inputting from the outside; and a display (not illustrated in the drawing) that can display and confirm key-inputted information. Both the key switch and the display are incorporated into the controller 20. Further, for example, the parameter input unit 24 may be formed of a circuit or the like that receives the above-mentioned parameters transmitted from an external terminal such as a notebook personal computer.

The parameter holding unit 25 holds rotational speed parameters and time parameters inputted to the parameter input unit 24. For example, a suitable memory may be adopted as the parameter holding unit 25. In this case, “holds” parameters means that the parameters “are stored” in the memory.

The central control unit 21 of the controller 20 instructs a rotational speed of the rotary body based on the rotational speed parameters (target amount R1_{T} of the first rotational speed and the target amount R2_{T} of the second rotational speed) held by the parameter holding unit 25, and suitably issues an instruction (command) to the motor driver 22 such that the motor driver 22 switches a rotational speed of the rotary body based on the time parameters (the first period T1, the second period T2, and the like) held by the parameter holding unit 25.

With such operations, it is possible to realize a rotation control of the rotary body that reflects the rotational speed parameters and the time parameters given from the outside.

### 2. Method of Driving Blood Pump According to Embodiment

Next, the method of driving the blood pump 10 is described.

The method of driving the blood pump 10 according to the embodiment is a method of driving the blood pump 10 by the left ventricular assist system 1 that includes the continuous-flow-type blood pump 10 where the blood pump 10 sucks and delivers blood by rotation of the rotary body (impeller 14) housed in the pump chamber 13. The terms used in the description of the method of driving the blood pump that are used in common with the terms used in the description of the configuration of the left ventricular assist system 1 according to the embodiment described above are assumed to form the common concept and hence, the description of these terms are omitted here.

FIG. 6 is a flowchart for describing the method of driving a blood pump according to the embodiment.

In the method of driving a blood pump according to this embodiment, in a normal operation mode S20, the blood pump 10 is driven such that a rotational speed of the rotary body is periodically switched between the “first rotational speed R1” and the “second rotational speed R2” that is larger than the first rotational speed R1 with a transition time required for switching the rotational speed set to substantially 0 (zero).

In the method, periodic switching of the rotational speed of the rotary body is asynchronous with a cardiac cycle of the user U of the left ventricular assist system 1, and the blood pump 10 is operated so as to make the rotary body rotate substantially only at rotational speeds of two values consisting of the first rotational speed R1 and the second rotational speed R2 (also see FIG. 5).

As illustrated in FIG. 6, prior to shifting to the normal operation mode S20, a parameter input step S10 may be performed.

The parameter input step S10 is a step where at least one “rotational speed parameter” out of the target amount R1_{T} of the first rotational speed and the target amount R2_{T} of the second rotational speed and at least one “time parameter” out of the first period T1, the second period T2 and one cycle P are inputted to the central control unit 21 prior to driving of the blood pump 10 in the normal operation mode S20.

In the normal operation mode S20, the central control unit 21 instructs a rotational speed of the rotary body based on the rotational speed parameters inputted in parameter input step S10, and performs switching of the rotational speed of the rotary body based on the time parameters inputted in the parameter input step S10.

3. Advantageous Effects Acquired by Left Ventricular Assist System 1 and Method of Driving a Blood Pump According to this Embodiment
(1) Case where this System is Applied to User who Originally has Weak Heartbeat or Pulses
(1-1) The left ventricular assist system 1 according to the embodiment is configured to control the blood pump 10 such that a rotational speed of the rotary body is periodically switched between the first rotational speed R1 and the second rotational speed R2.

When this system having such a configuration is applied to a user U who originally has weak heartbeat or has weak pulses because of some causes, with respect to factors that change a blood flow or a blood pressure in a systemic circulation system of the user U, the factor that changes a blood flow or a blood pressure due to a change in rotational speed of the blood pump 10 (auxiliary route 55) is superior to the factor that changes a blood flow or a blood pressure due to the pulsation of the own heart 80 of the user U (original route 95) if either one of these factors is to be chosen (see FIG. 3). Accordingly, in a case where this system is applied to such a user U, in association with periodic switching of the rotational speed of the blood pump 10 between the first rotational speed R1 and the second rotational speed R2, the blood flow or the blood pressure changes and the own heart 80 of the user U pulsates at a substantially equal cycle P also in the systemic circulation system of the user U. In this manner, the pulsation is artificially made also in the systemic circulation system and hence, the user who has originally weak pulses generated by the own heart 80 can enjoy “physiological phenomenon expected by pulses” described in the chapter “Description of the Related Art”.

Basically, the periodic switching of the rotational speed of the rotary body (cycle of the change in rotational speed) is asynchronous with the cardiac cycle of the user U of the left ventricular assist system 1. However, in a case where the user U originally has weak heartbeat or pulses, as described above, the change in rotational speed of the blood pump 10 is superior to the pulses of the own heart 80. Accordingly, irrelevant to whether the cycle of the change in rotational speed is asynchronous or synchronous with the cardiac cycle of the user U, the user U can reasonably enjoy a “physiological phenomenon expected by pulses” because of a change in rotational speed of the blood pump 10.

(1-2) According to the left ventricular assist system 1, in the second period T2 where the rotary body rotates at the second rotational speed R2, a blood flow (eventually, a pressure) in the auxiliary route 55 is increased compared to the blood flow and the pressure during the first period T1. Accordingly, it is possible to expect the generation of a blood flow that flows backward in a direction toward the aortic valve 86 from an area where the auxiliary route 55 and the original route 95 merge to each other (an area in the vicinity of a connecting point where the artificial blood vessel 41 is connected to the aorta 90). This backward flow moves the aortic valve 86 in the direction that the aortic valve 86 is temporarily closed and hence, blood in the inside of the aorta 91 (for example, an area in the vicinity of an area indicated by symbol 91a) and blood in the left ventricle 82 that stagnates in the vicinity of the aortic valve 86 are moved. As a result, the blood flows in the direction that the stagnation of the blood in the vicinity of the aortic valve is eliminated.
(1-3) In the left ventricular assist system 1, the controller 20 is configured to operate the blood pump 10 such that the rotary body rotates substantially only at rotational speeds of two values consisting of the first rotational speed R1 and the second rotational speed R2 with a transition time required for switching the rotational speed set to substantially 0 (zero).

That is, as described above, the controller 20 is configured such that the operation of the blood pump 10 can be realized by allowing the central control unit 21 to merely issue an instruction (issuing of a command, outputting of a control signal or the like) of a change in rotational speed between two values in a simply discrete manner to the motor driver 22. In other words, the controller 20 does not require a complicated algorism or a complicated circuit that is required to perform the following operations. These operations that require the complicated algorithm and the complicated circuit include: an operation where the blood pump 10 is operated by selecting and switching the large number of variations between rotational speeds of three or more values; a ramp driving that requires a transitional time (transition time) in switching a rotational speed and has a speed gradient, and an operation where the blood pump 10 is operated such that peaks are instantaneously generated in a change pattern of a rotational speed so as to simulate healthy pulses (steep change in rotational speed).

This advantageous effect can be directly acquired also in the manners of operation and advantageous effects described in the following paragraph (2) and the succeeding paragraphs.

As described in paragraphs (1-1) to (1-3), the left ventricular assist system 1 according to the embodiment can minimize the stagnation of blood while having the simple configuration. Further, the left ventricular assist system 1 enables a user U who originally has weak pulses generated by own heart 80 to enjoy a “physiological phenomenon expected by pulses”.

(2) Particularly, in a case where this system is applied to the user U who originally has weak heartbeat or pulses, when the user U of the left ventricular assist system 1 is at rest, the controller 20 is configured to operate the blood pump 10 such that the number of appearances of the second period T2 becomes a predetermined number within a range of 40 times to 100 times per minute. The controller 20 is configured to operate the blood pump 10 such that a duty ratio of the second period T2 in one cycle P falls within a range of 20% to 40%. The second rotational speed R2 can be set such that a blood flow (a total flow rate TF) in the aorta 90 of the user U of the left ventricular assist system 1 falls within a range of 10 L to 30 L per minute as a peak flow amount. Further, the second rotational speed R2 is a rotational speed set by taking into account a desired peak flow amount of a blood flow (a total flow rate TF) in the aorta 90 of the user U of the left ventricular assist system 1, and the first rotational speed R1 is a rotational speed set such that a blood flow (a total flow rate TF) in the aorta 90 of the user U takes a value in the vicinity of 0 (zero).

In this manner, by making the behavior of a change in rotational speed by the blood pump 10 approximate the healthy pulsation (function of a heart) of a person in general, even in a systemic circulation system of a user U who originally has weak heartbeat or pulses, it is possible to build pseudo pulses similar to pulses of a healthy person. Accordingly, such a user U can enjoy “a physiological phenomenon expected by pulses”.

(3) As the blood pump 10, it is desirable to adopt a blood pump that has a delivery ability of 15 L or more per minute when the rotary body is rotated at the second rotational speed R2 when an aorta-left-ventricle pressure difference that is a difference between an inner pressure P3 of the aorta 90 and an inner pressure P2 of the left ventricle 82 is 0 (zero).

By adopting the blood pump 10 that can acquire a high peak flow and by applying the blood pump 10 to the user U, it is expected that a change in internal pressure in the inside 91 of the aorta 90 is increased. Accordingly, the blood pump 10 can realize a function close to healthy pulsations (function of heart) of a person in general.

(4) Case where System According to Embodiment is Applied to User whose Pulses can be Relatively Clearly Confirmed

Next, the manner of operation and the advantageous effects acquired in a case where the left ventricular assist system 1 according to the embodiment is applied to a user whose pulses in the systemic circulation system can be relatively clearly confirmed.

First, as can be also understood from FIG. 3, an internal pressure in the inside 91 of the aorta 90 is a pressure generated by making a pressure via the original route 95 generated by pulsations of the own heart 80 is made to overlap with a pressure via the auxiliary route 55 generated by the blood pump 10.

The pressure via the original route 95 changes along a cardiac cycle of the own heart 80. On the other hand, a pressure via the auxiliary route 55 changes by periodic switching of a rotational speed of the blood pump 10.

In the left ventricular assist system 1 according to the embodiment, the periodic switching of a rotational speed of the rotary body of the blood pump 10 is asynchronous with a cardiac cycle of the user U. Accordingly, “waviness” occurs in the internal pressure generated in the inside 91 of the aorta 90. In other words, the combination of respective periods that form the cardiac cycle of the own heart 80 (roughly classified into a systolic period and a diastolic period) and the respective periods (first period T1, second period T2) that form the cycle of the change in rotational speed of the blood pump 10 changes one after another.

(4-1) For example, a case is estimated where the blood pump 10 falls within the first period T1 (low rotational speed) at timing of the systolic period (more specifically, the ejection period) of the own heart 80.

This period is a period where a blood delivery ability to deliver blood from the auxiliary route 55 to the inside 91 of the aorta 90 is low and hence, a pressure applied to the inside 91 of the aorta 90 from the auxiliary route 55 is low. Also because of this factor, in the original route 95, the situation is changed in the direction that a pressure difference (P2-P3) between the pressure P3 in the inside 91 of the aorta 90 and the pressure P2 in the left ventricle 82 is increased and hence, the aortic valve 86 can be firmly opened to a full movable range. Accordingly, at the timing that such combination is established, the situation moves in the direction that the stagnation of blood in the vicinity of the aorta 86 is eliminated.

To describe the above-mentioned periods for a reference purpose, “ejection period” is a period where a wall of the left ventricle 82 pushes out blood in the ventricle, and is also a period where the aortic valve 86 opens in a state where the mitral valve 85 is closed and blood is delivered to the aorta 90.

(4-2) For example, a case is estimated where the blood pump 10 is in the second period T2 (high rotational speed) at timing that the own heart 80 is in an isovolumic relaxation (the mitral valve 85 and the aortic valve 86 being closed), and the blood pump 10 is in the first period T1 (low rotational speed) at timing that the own heart 80 is in a diastolic filling period (the mitral valve 85 being opened) that comes immediately after the isovolumic relaxation.

In this case, in the isovolumic relaxation, the delivery of blood in the left ventricle 82 via the auxiliary route 55 is enhanced by the blood pump 10 so that a wall of the heart in the vicinity of the left ventricle 82 is contracted more compared to a usual time. When the own heart 80 is shifted to the diastolic filling period from the state where the wall of the heart in the vicinity of the left ventricle 82 contracts, as a reaction of such a shift, an amount of blood larger than an amount of blood in a usual cardiac cycle flows into the left ventricle 82. Further, in cooperation with a state of the blood pump 10 where the blood pump 10 is rotated at a low rotational speed so that delivery ability is lowered, an amount of blood larger than an amount of blood in a usual state is filled in the left ventricle 82. That is, the wall of the heart in the vicinity of the left ventricle 82 is stretched (a state where the left ventricle 82 is expanded compared to a usual state). Accordingly, blood that may be stagnated in a part of the inside of the left ventricle 82 starts its movement. That is, the blood acts in the direction that the stagnation of the blood is eliminated.

(4-3) With respect to the combination of: the systolic period and the diastolic period of the own heart 80; and the period (the first period T1) in which the blood pump 10 rotates at the first rotational speed R1 and the period (the second period T2) in which the blood pump 10 rotates at the second rotational speed R2, various other combinations are considered besides the above-mentioned combination, and the stagnation of blood can be eliminated by these respective combinations.

In the conventional left ventricular assist system, in general, the circulation of blood is assisted by operating a blood pump by rotating an impeller at a fixed rotational speed. Accordingly, the aorta 90, the aortic valve 86, the left ventricle 82 and the like move in accordance with a fixed routine.

On the other hand, in the left ventricular assist system 1 according to the embodiment, as described in the above-mentioned (3-1) to (3-4), the periodic switching of the rotational speed of the rotary body is asynchronous with a cardiac cycle of the own heart 80 of the user U. Accordingly, the combination of the respective periods that form a cardiac cycle of the own heart 80 and the respective periods that form the cycle of a change in rotational speed of the blood pump 10 changes one after another. Accordingly, the aorta 90, the aortic valve 86, the left ventricle 82 and the like are placed under various states and take various movements. As a result, the blood acts in the direction that the stagnation of blood in the aorta 90, the aortic valve 86, the left ventricle 82 and the like is eliminated. Accordingly, the occurrence of a thrombus can be suppressed more effectively.

In the same manner as the above-mentioned (1) to (3), the left ventricular assist system 1 according to the embodiment is configured such that the rotary body is periodically rotated substantially only at the rotational speeds of two values consisting of the first rotational speed R1 and the second rotational speed R2 with the transition time required for switching the rotational speed set to substantially 0 (zero). Accordingly, it is possible to easily build this system.

As a result, according to the left ventricular assist system 1 of the embodiment, the stagnation of blood can be suppressed with the simple configuration.

(5) In the left ventricular assist system 1 according to the embodiment, the controller 20 includes: the parameter input unit 24 that inputs “rotational speed parameters” and “time parameters”; and the parameter holding unit 25 that holds the rotational speed parameters and the time parameters. The controller 20 is configured to instruct a rotational speed of the rotary body based on the rotational speed parameters held in the parameter holding unit 25, and is configured to perform switching of the rotational speeds of the rotary body based on the time parameters held by the parameter holding unit 25.

With such a configuration, for example, in response to a change in a state of the user U himself/herself or a change in an environment where the user U exists, the rotational speed parameters and the time parameters are suitably and variably set or changed from the outside, and the blood pump 10 can be operated by reflecting such setting or change in the operation. Accordingly, the left ventricular assist system 1 can assist the function of the own heart 80 of the user U more finely and suitably.

The essential parts of the method of driving a blood pump according to the embodiment are substantially equal to the essential parts of the left ventricular assist system 1 according to the embodiment. Accordingly, the method of driving a blood pump can acquire substantially the same advantageous effects acquired by the left ventricular assist system 1.

Further, the technical features described in this specification (the embodiment and modifications described later) can be incorporated into both the left ventricular assist system 1 and the method of driving a blood pump.

Although the present invention has been described based on the above-mentioned embodiment, the present invention is not limited to the above-mentioned embodiment. Various modifications are conceivable in various modes without departing from the gist of the present invention, and the following modifications are also conceivable, for example.

(1) In the embodiment, the description has been made by estimating mainly the case where the user U is at rest for an exemplifying purpose. However, the present invention is not limited to such a case. For example, when the user is making a physical exercise, the time parameters may be set such that the number of times of appearances of the second period T2 becomes the number of times within a range of approximately 70 to 120 times per minute so that a duty ratio of the second period T2 in one cycle P falls within a range of 30% to 50%. In this manner, the rotational speed parameters and the time parameters can be suitably and variably set and changed in response to a change in a state of the user U himself/herself, a change in an environment where the user U exists or the like.
(2) In the embodiment, the description has been made by taking the case of the left ventricular assist system where the blood pump 10 is operated by setting the number of appearances of the second period T2 per 1 minute and a duty ratio of the second period T2 in 1 cycle P such that the number of pulses becomes substantially equal to the number of pulses of a heathy person. However, the present invention is not limited to such a case.

For example, in the left ventricular assist system, the blood pump 10 may be operated by setting the second period T2 to approximately 55 seconds, the first period T1 to approximately 5 seconds (the number of appearances of the second periods T2 being set to approximately 1 time per 1 minute, the number of appearances of the first periods T1 being set to approximately 1 time per 1 minute, and a duty ratio of the second period T2 in 1 cycle P being set to a value that falls approximately within a range of 80% to 90%). That is, the blood pump 10 may be configured such that a rotational speed may be changed with a cycle that is far longer than a cycle of pulses of a healthy person.

By adopting such a configuration, the blood pump 10 is rotated at the second rotational speed R2 that is a high rotational speed during a period that occupies a most portion of a cardiac cycle of a person so as to strongly assist the left ventricle. On the other hand, sometimes, the blood pump 10 is rotated at the first rotational speed R1 that is a low speed so that a flow rate of the original route 95 can be temporarily increased relative to a flow rate of the auxiliary route 55. Accordingly, the aortic valve 86 can be easily opened during the first period T1 where the rotary body is rotated at the first rotational speed R1 and hence, the situation can be directed in the direction that the stagnation of blood in the vicinity of the aortic valve 86 can be eliminated.

1. A left ventricular assist system of an implantable type comprising at least:
a continuous-flow-type blood pump configured to suck and deliver blood by rotation of a rotary body housed in a pump chamber; and
a controller connected to the blood pump and configured to control the rotation of the rotary body of the blood pump, wherein
in a normal operation mode, the controller is configured to control the blood pump such that a rotational speed of the rotary body is periodically switched between a “first rotational speed” and a “second rotational speed” that is larger than the first rotational speed with a transition time required for switching the rotational speed set to substantially 0 (zero), and
periodic switching of the rotational speed of the rotary body is asynchronous with a cardiac cycle of a user of the left ventricular assist system, and the blood pump is operated so as to make the rotary body rotate substantially only at rotational speeds of two values consisting of the first rotational speed and the second rotational speed.

2. The left ventricular assist system according to claim 1, wherein defining a sum of a “first period” that is a period during which the rotary body is rotated at the first rotational speed and a “second period” that is a period during which the rotary body is rotated at the second rotational speed as one cycle,
the controller includes:
a parameter input unit that inputs at least one parameter of a target amount of the first rotational speed and a target amount of the second rotational speed that form “rotational speed parameters”, and at least one parameter out of the first period, the second period and the one cycle that form “time parameters”; and
a parameter holding unit that holds the rotational speed parameters and the time parameters, and
a rotational speed of the rotary body is instructed based on the rotational speed parameters held in the parameter holding unit, and switching of the rotational speed of the rotary body is performed based on the time parameters held by the parameter holding unit.

3. The left ventricular assist system according to claim 1, wherein defining a sum of a “first period” that is a period during which the rotary body is rotated at the first rotational speed and a “second period” that is a period during which the rotary body is rotated at the second rotational speed as one cycle,
a duty ratio of the second period in the one cycle falls within a range of 20% to 40%.

4. The left ventricular assist system according to claim 1, wherein defining a sum of a “first period” that is a period during which the rotary body is rotated at the first rotational speed and a “second period” that is a period during which the rotary body is rotated at the second rotational speed as one cycle,
when the user of the left ventricular assist system is at rest, the blood pump is operated such that the number of appearances of the second period becomes a predetermined number within a range of 40 times to 100 times per minute

5. The left ventricular assist system according to claim 1, wherein the second rotational speed is a rotational speed that is set by taking into account a desired peak flow amount of a blood flow in an aorta of the user of the left ventricular assist system, and
the first rotational speed is a rotational speed that is set such that a blood flow in the aorta of the user becomes a value in the vicinity of 0 (zero).

6. The left ventricular assist system according to claim 1, wherein the second rotational speed is a rotational speed that is set such that a blood flow in the aorta of the user of the left ventricular assist system falls within a range of 10 L to 30 L per minute as a peak flow amount.

7. The left ventricular assist system according to claim 1, wherein the blood pump has a delivery ability of 15 L or more per minute when the rotary body is rotated at the second rotational speed when an aorta-left-ventricle pressure difference that is a difference between an inner pressure of an aorta and an inner pressure of a left ventricle is 0 (zero).

8. A method of driving a blood pump in a left ventricular assist system that includes a continuous-flow-type blood pump configured to suck and deliver blood by rotation of a rotary body housed in a pump chamber, wherein
in a normal operation mode, the blood pump is operated such that a rotational speed of the rotary body is periodically switched between a “first rotational speed” and a “second rotational speed” that is larger than the first rotational speed with a transition time required for switching the rotational speed set to substantially 0 (zero), and
periodic switching of the rotational speed of the rotary body is asynchronous with a cardiac cycle of a user of the left ventricular assist system, and the blood pump is operated so as to make the rotary body rotate substantially only at rotational speeds of two values consisting of the first rotational speed and the second rotational speed.

9. The method of driving a blood pump according to claim 8, wherein
defining a sum of a “first period” that is a period during which the rotary body is rotated at the first rotational speed and a “second period” that is a period during which the rotary body is rotated at the second rotational speed as one cycle,
prior to shifting to the normal operation mode, a parameter input step is performed where at least one “rotational speed parameter” out of a target amount of the first rotational speed and a target amount of the second rotational speed and at least one “time parameter” out of the first period, the second period and one cycle are inputted prior to driving of the blood pump in the normal operation mode, and
in the normal operation mode, a rotational speed of the rotary body is instructed based on the rotational speed parameters inputted in the parameter input step, and switching of the rotational speed of the rotary body is performed based on the time parameters inputted in the parameter input step.
